(19) United States
(12) Patent Application Publication
(10) Pub. No.: US 2003/0215564 A1
(43) Pub. Date: Nov. 20, 2003

(54) Method and apparatus for coating an endoprosthesis

(76) Inventors: Phillip F. Heller, Baltimore, MD (US); Alan W. Heldman, Elkridge, MD (US); Jeffrey P. Foehlich, Baltimore, MD (US); Dong-Woon Kim, Cheongiu Chungbuk (KR)

Correspondence Address: KLARQUIST SPARKMAN, LLP, 121 SW SALMON STREET, SUITE 1600, PORTLAND, OR, 97204, US

(21) Appl. No.: 10/181,309

(22) PCT Filed: Jan. 18, 2001
(86) PCT No.: PCT/US01/01783

(51) Int. Cl. B05D 1/40; A61F 2/06

(52) U.S. Cl. 427/2.25; 118/57; 623/1.11; 623/1.42

(57) ABSTRACT

Methods and apparatus are disclosed for coating endoprostheses. The methods use distributive forces such as centrifugal force or vibration to distribute a bioactive liquid over the surface of the endoprosthesis. In some embodiments, the endoprosthesis is elongated along the longitudinal axis, and is substantially tubular, such as a stent. In other embodiments, the force is applied by rotating the endoprosthesis at speeds between 100-100,000 RPM, or by vibrating it at frequencies between about 10 Hz and about 200,000 Hz. The invention also includes a device for providing a predictable coating on the surface of an endoprosthesis, which includes means for applying bioactive liquid to the endoprosthesis, and means for applying centrifugal force or vibration to distribute the bioactive liquid. The invention also includes endoprostheses that have been subjected to centrifugal force to distribute a bioactive liquid coating layer.

### TECHNICAL FIELD

This invention generally relates to the coating of endoprostheses and, more particularly, to coating endoprostheses such as stents with therapeutic substances.

### BACKGROUND

Endoprostheses are synthetic or natural substitutes for any part of the body. They may be any material or article of manufacture that is put in or attached to a body to enhance, assist or replace the function of any organ or tissue. Currently they are used for definitive or palliative therapy in orthopedic, gastrointestinal, cardiovascular and other conditions, and their range of uses continues to expand.

A family of endoprostheses known as stents has proven to be particularly effective. Generally, a stent is a substantially tubular medical device designed to prop open a hollow organ, such as a blood vessel, bile duct, esophagus, etc. Stents have markedly improved therapy for a number of human diseases, such as restenosis and abrupt vessel closure following balloon angioplasty (U.S. Pat. No. 4,733,665), biliary obstruction (U.S. Pat. No. 5,776,160) and esophageal cancer (U.S. Pat. No. 5,876,448).

The utility of endoprostheses may be enhanced by coating them with a variety of substances, such as polymers, drugs, or genetic material. The coating may improve the function of the endoprosthesis, for example by making it more wear-resistant, increasing positional stability, or enhancing its resistance to microbial colonization. Examples include: U.S. Pat. No. 5,891,506 (coating medical devices to enhance biocompatibility); U.S. Pat. No. 5,152,794 (coating orthopedic endoprostheses to improve durability); U.S. Pat. No. 5,770,255 (coating endopiostheses with antibiotics to improve their resistance to infection). Alternatively, the coating may contain substances designed to treat the underlying disease, for example an arterial stent coated with medications designed to inhibit vascular disease (see, for example, U.S. Pat. No. 5,554,182, coating vascular stents with fibrin and heparin to reduce restenosis after angioplasty).

Whatever the coating material(s), it is often important to distribute the coating evenly. In some instances an asymmetric coating may be preferred, as in U.S. Pat. No. 5,876,448, which describes an esophageal stent uncoated on its ends, so that it is easier to anchor in its proper position. In either case, however, consistency and reproducibility are often desirable. In addition, in some circumstances it may be important to control thickness. A coating that is too thick may be unduly prone to cracking or flaking, or have other flaws. A coating that is too thin may result in a substandard endoprosthesis, for example by failing to deliver adequate amounts of a therapeutic substance, by being bioincompatible, or other reasons.

In some circumstances, an operator might wish to reproduce a successful coating result. For example, an operator may learn that coating an endoprosthesis with a substance in a defined amount and distribution has a favorable therapeutic effect. That same operator, or another operator, may wish to replicate the successful coating as closely as possible. To achieve this, a device and method is needed that allows precise and accurate reproduction of results between different operators, even if they are working at different times and in different locations.

In some circumstances, it may be desirable to conserve the coating material. For example, an endoprosthesis may be coated with a rare and valuable chemotherapeutic agent. In this circumstance, one would want to save as much of the excess coating as possible, to use on another endoprosthesis or otherwise conserve and reuse it.

U.S. Pat. No. 5,234,457 describes impregnated stents in which liquid gelatin is poured from a container onto the stents while they are being rotated. U.S. Pat. No. 5,897,911 describes mounting stents on a pin that maintains a defined separation between stent and pin; the assembly is then dipped in liquid polymer. U.S. Pat. No. 5,980,972 describes spraying the stent, first with polymer, then with a therapeutic substance.

What is needed is a device and method for coating endoprostheses that controls the amount, distribution, and/or thickness of coating substance(s) in an operator-independent, precise and accurate manner, and which may be capable of conserving the coating material as much as possible.

### SUMMARY

A method and apparatus are disclosed for coating endoprostheses that control the amount, distribution, and thickness of coating substance(s) in an operator-independent, precise and accurate manner, while conserving the coating material.

The method applies a distributing force, such as centrifugal force or high frequency vibration to distribute a bioactive liquid over the surface of the endoprosthesis. The bioactive liquid may contain a polymer, a therapeutic substance such as a drug or genetic material, or any other desired substance. In some embodiments, the endoprosthesis is surrounded by a shield during application of centrifugal force or vibration, to collect excess liquid as it is removed from the endoprosthesis. In some embodiments, the endoprosthesis is elongated along the longitudinal axis, and is substantially tubular, such as a stent.

The invention also includes a device for providing a predictable coating on the surface of an endoprosthesis, which includes means for applying bioactive liquid to the endoprosthesis, and means for applying a distributing force, such as centrifugal force or high frequency vibration to distribute the bioactive liquid over the surface of the endoprosthesis. The invention also includes endoprostheses that have been subjected to the distributing force, such as centrifugal force or vibration to distribute a bioactive liquid coating layer.

As is apparent from the foregoing, the present invention includes many different advantages and permutations. The foregoing and other features and advantages of the invention will become more apparent from the following detailed description of disclosed embodiments, which proceeds with respect to the accompanying drawings.

### BRIEF DESCRIPTION OF THE FIGURES

FIG. 1 is a perspective side view of a stent mounted on a mandrel, and poised above a vat containing a bioactive liquid into which the stent is to be introduced.

FIG. 2 is a perspective side view of a stent mounted on a mandrel which has been inserted into a shield to collect bioactive liquid as it is removed from the stent by centrifugal force as the mandrel is rotated.

FIG. 3 is a cross-sectional view along section lines 3-3 in FIG. 2, illustrating how the shield collects excess liquid.

FIG. 4 is a view similar to FIG. 2, but showing a sleeve inserted between the mandrel and stent.

FIG. 5 is a cross-sectional view taken along section lines 5-5 in FIG. 4.

FIG. 6 is a perspective view showing bioactive liquid applied to the stent by spraying.

FIG. 7 is a perspective side view of a stent mounted on a catheter, and attached to a mandrel.

FIG. 8 is a perspective side view of a catheter-stent assembly, with the catheter-stent assembly partially contained within a catheter containment device.

### DETAILED DESCRIPTION OF SEVERAL ILLUSTRATIVE EMBODIMENTS

The present disclosure includes a method of coating an endoprosthesis by applying a bioactive liquid to its surface, and applying a distribution force, such as centrifugal force or vibration to the endoprosthesis. The centrifugal force or vibration distributes the bioactive liquid over the surface of the endoprosthesis. In particular embodiments, the coated surface is at least an outer surface of the endoprosthesis, and the centrifugal force or vibration distributes the bioactive liquid substantially evenly over the outer surface. The endoprosthesis may be elongated along a longitudinal axis. Centrifugal force is applied by rotating the elongated endoprosthesis substantially around the longitudinal axis, or the elongated prosthesis is vibrated. In certain embodiments, the endoprosthesis is substantially symmetric with respect to the longitudinal axis, or is substantially tubular.

The bioactive liquid may be applied by immersion, spraying, or pouring, or any combination of these or other methods. In some examples, the bioactive liquid is applied substantially completely to a therapeutic substrate surface across which it is desired that a bioactive substance will act (for example, to substantially completely cover an outer surface of a prosthesis that will contact tissue, such as an outer surface or an endovascular stent that will be in contact with a vascular wall after implantation. In some embodiments a shield surrounds the endoprosthesis while centrifugal force or vibration is applied to it, to collect the bioactive liquid removed from the endoprosthesis by centrifugal force or vibration. The excess bioactive liquid so obtained may be used to coat another endoprosthesis, or otherwise conserved and put to a different use. The shield may be a tubular member, and the bioactive liquid may be a therapeutic substance such as antithrombotic agents, antiplatelet agents, anti-inflammatory agents, antibiotics, anti-angiogenic agents, angiogenesis-promoting agents, antioxidants, antiproliferative agents, anti-atherogenic agents, vasoactive agents, photoactivated agents, photosensitizing agents, radiation sensitizing agents, acoustic energy sensitizing agents, radioactive agents, imaging agents, or hormones. For example, the therapeutic substance may be a taxane, such as paclitaxel and its analogs.

The endoprosthesis may be a stent, for example a vascular stent. However, the principles of the invention can be applied to a broad variety of prosthetic devices, such as intra-articular prostheses, gastrointestinal stents, and endobronchial stents. Examples of intra-articular prostheses include artificial hip joints and artificial knees. Examples of gastrointestinal stents include esophageal or biliary stents.

In some embodiments, the stent is mounted on an insertion device prior to coating with bioactive liquid. For example, the stent may be mounted on a catheter prior to coating with liquid, and the catheter may have an inflatable distal end upon which the stent is mounted. The catheter may be a vascular catheter, for example a balloon angioplasty catheter. The insertion device upon which the stent is mounted may be attached to a mandrel which is rotated or vibrated, or may be at least partly contained within a catheter containment device which is rotated our vibrated.

The centrifugal force or vibration may be applied to an endoprosthesis by inserting a mandrel into the endoprosthesis and rotating or vibrating the mandrel, for example with a motor. In certain disclosed examples, the centrifugal force is applied by rotating the endoprosthesis (or in some embodiments the mandrel on which the endoprosthesis is mounted) at 100-100,000 RPM; more particularly, 1,000-50,000 RPM; and even more particularly, 10,000-35,000 RPM. In other disclosed examples, force is applied to the endoprosthesis by vibrating the endoprosthesis (or in some embodiments the mandrel on which the endoprosthesis is mounted), for example at frequencies between about 10 Hz and about 200,000 Hz; more particularly, between about 40 Hz and about 100,000 Hz; and even more particularly, between about 50 Hz and about 40,000 Hz. In other particular examples, the mandrel may be vibrated at frequencies in the subsonic, sonic, or ultrasonic range. In particular embodiments, a sleeve is placed between the mandrel and endoprosthesis, of sufficient thickness to firmly engage the endoprosthesis and rotate it with the mandrel.

In yet other particular embodiments, substantially the same centrifugal force or vibration is applied to different endoprostheses (such as distinct but substantially identical endoprostheses) to improve uniformity in the amount and distribution of the liquid on the endoprostheses. In some embodiments, the viscosity of the liquid applied to multiple endoprostheses is substantially constant, and the endoprostheses are rotated or vibrated at similar speeds or frequencies, thereby increasing uniformity of surface distribution.

Also disclosed are methods of providing a predictable coating of a therapeutic substance on an external surface of multiple elongated hollow vascular stents, by inserting a mandrel into each of the hollow stents and securing each stent to the mandrel, and rotating the stents at a substantially uniform speed of rotation that provides a substantially uniform coating of the therapeutic substance on the surface.

Also disclosed are devices for coating an endoprosthesis, having a means for applying a bioactive liquid to the surface of the endoprosthesis, and a means for applying centrifugal force or vibration to the endoprosthesis to distribute the bioactive liquid over the surface. The device may also include a means for associating the endoprosthesis with a force applicator, such as a driving member capable of rotating or vibrating the endoprosthesis, a means for applying the force, for example by rotating or vibrating the endoprosthesis to distribute the bioactive liquid over the surface of the endoprosthesis, and a means for collecting bioactive liquid that leaves the surface of the endoprosthesis during rotation. In other embodiments, the device includes a driver for rotating or vibrating the endoprosthesis, and a shield surrounding the endoprosthesis for collecting liquid that leaves the surface of the endoprosthesis during rotation or vibration. The device may also include a source of the bioactive liquid.

Also disclosed are endoprostheses having a surface to which a bioactive liquid has been applied and distributed by the distribution force, for example the centrifugal force or vibration. In particular embodiments, the liquid is a therapeutic substance. The endoprostheses may be stents, for example vascular stents. The stents may be substantially hollow and elongated about an axis of elongation, and may be coated by inserting a mandrel into each of the hollow stents, securing the stent to the mandrel, and rotating or the mandrel and stent at a preselected speed of rotation or frequency of vibration.

### Explanations of Terms

“Angiogenesis-promoting agent” refers to any substance that directly or indirectly promotes the formation of blood vessels, or helps to maintain existing blood vessels.

An “antioxidant” is a substance that significantly delays or prevents oxidation of the substrate biological molecules. Antioxidants can act by scavenging biologically important reactive free radicals or other reactive oxygen species (O₂^{—}, H₂O₂, OH, HOCl, ferryl, peroxyl, peroxynitrite, and alkoxyl), or by preventing their formation, or by catalytically converting the free radical or other reactive oxygen species to a less reactive species.

“Anti-angiogenic agent” refers to any substance that directly or indirectly opposes or limits the formation of blood vessels, or promotes regression of existing blood vessels.

“Anti-atherogenic agent” refers to any agent that directly or indirectly opposes or limits formation of atheromatous deposits, especially on blood vessel walls, or any agent that limits or otherwise reduces blood vessel injury or inflammation.

“Anti-migratory” agent refers to any agent that limits the ability of a cell to move.

“Antiproliferative agent” refers to any agent that inhibits, reduces, slows, or otherwise limits the growth of a cell.

An “antibiotic” is any substance that directly or indirectly kills, inhibits the growth of, or otherwise impairs the function of, any living microorganism, including but not limited to bacteria, viruses, mycoplasma, chlymadia, rickettsiae, fungi, molds, yeasts, protozoan, parasitic and helminthic species.

An “anti-inflammatory agent” means any substance that directly or indirectly inhibits, opposes, or reverses any step in the process of inflammation, including but not limited to limitation, opposition, or reversal of: vasodilation; increased vascular permeability; exudation of fluids; expression of adhesion molecules, selecting, and other molecules that attract inflammatory cells; adhesion, infiltration or retention of inflammatory cells; release of growth factors, cytolines, or chemokines; or expression of genes associated with the inflammatory response.

An “antiplatelet agent” means any substance that directly or indirectly inhibits, opposes, or reverses any aspect of platelet function, including but not limited to inhibition, opposition, or reversal of: adhesion; aggregation; activation; shape change; conformational change in glycoprotein IIb/IIIa complexes; binding of fibrinogen to glycoprotein IIb/IIIa complexes; release of granule contents; secretion; changes in levels of cyclic nucleotides; enhancement of calcium influx or mobilization of calcium from intracellular stores; hydrolysis of membrane phospholipids; phosphorylation or dephosphorylation of intracellular proteins; physiologic function of intracellular enzymes; or generation of arachadonic acid, endoperoxides, or thromboxanes.

An “antithrombotic agent” means: (1) any substance that directly or indirectly limits, opposes, or reverses any step in the process of blood clot formation or maintenance, including but not limited to inhibition, opposition, or reversal of contact system or tissue factor-mediated system activation; activation of any clotting factor; thrombin generation; conversion of fibrinogen into fibrin; generation of fibrin polymers; or crosslinking of fibrin polymers; or (2) any substance that promotes, enhances or mimics the action of physiologic anticoagulants such as heparin, antithrombin, protein C, protein S, or tissue factor pathway inhibitor; (3) any substance that directly or indirectly promotes or enhances any step in the process of thrombolysis, including but not limited to (a) promotion or enhancement of urinary plasminogen activator or tissue plasminogen activator action, conversion of plasminogen into plasmin, or fibrin degradation; or (b) inhibition, opposition, or reversal of plasminogen activator inhibitors or alpha-2 plasmin inhibitors.

A “photoactivated agent” is a bioactive agent with a desired biological activity that is partially or completely activated by exposure to visible or ultraviolet light.

A “photosensitizing agent” is a bioactive agent that increases the sensitivity of a cell or tissue to the effects of visible or ultraviolet light. A “radiation sensitizing agent” is a bioactive agent that increases the sensitivity of a cell or tissue to the effects of exposure to ionizing radiation. An “acoustic-energy sensitizing agent” is a bioactive agent that increases the sensitivity of a cell or tissue to the effects of exposure to subsonic, sonic, or ultrasonic vibrations.

A “radioactive agent” is any agent that emits ionizing radiation.

An “imaging agent” is a bioactive agent that enhances, simplifies, promotes or improves detection or localization of a bioprosthesis in a subject.

“Bioactive” means having a desired biological activity such as detectability, or a therapeutic or pharmaceutical effect, or even an absence of an effect (as in a biologically inert material which inhibits a physiologic response to a substrate).

“Biocompatible” means the ability to exist alongside or within living organisms without causing unacceptable or substantial harm.

“Bioincompatible” means the tendency to harm living organisms when existing alongside or within them.

A “catheter” is a substantially tubular and generally flexible device that carries fluids into or out of the body. A vascular catheter is a catheter that may be inserted into a blood vessel, for example a vein or artery.

“Centrifugal force” means the apparent force which seems to pull an object outward when the object is moved along a curved path.

An “endoprosthesis” is any synthetic or natural substitute for any part of the body, or any material or article of manufacture which is put in or attached to a body to enhance, assist or replace the function of any organ, tissue or cell, or prevent dysfunction of any organ or tissue. Some examples of endoprostheses include:

Cardiovascular endoprostheses: intravascular stents (U.S. Pat. No. 5,102,417), vessel occluders (U.S. Pat. No. 5,382,261), vena cava filters (U.S. Pat. No. 5,382,261), aortic intraluminal prostheses (U.S. Pat. No. 5,219,355), pacemakers and leads (U.S. Pat. No. 5,957,957), implantable defibrillators and leads (U.S. Pat. No. 4,693,253), prosthetic heart valves (U.S. Pat. No. 5,919,226).

Gastrointestinal endoprostheses: esophageal stents (U.S. Pat. No. 5,667,273), biliary stents (U.S. Pat. No. 5,776,160), intestinal stents (U.S. Pat. No. 4,057,065), implantable pulse generators (U.S. Pat. No. 5,995,872).

Genioturinary endoprostheses: ureteral stents (U.S. Pat. No. 5,681,274), urethral catheters (U.S. Pat. No. 4,148,319), implantable penile prostheses (U.S. Pat. No. 4,187,839), radioactive implants (U.S. Pat. No. 5,295,245).

Orthopedic ezdoprostheses: artificial joint replacements (U.S. Pat. No. 4,950,300), bone screws (U.S. Pat. No. 4,854,311).

Pulmonary endoprostheses: endotracheal tubes (U.S. Pat. No. 4,327,721), endobronchial stents (U.S. Pat. No. 4,248,221), tracheal stents (U.S. Pat. No. 5,480,431).

“Genetic material,” as used herein, means any material that contains any nucleic acid, oligonucleotide, or derivative thereof, that expresses or is capable of expressing any protein or polypeptide, or that affects expression of any gene, or is capable of affecting the expression of any gene.

A “hormone” means any chemical substance produced by cells in the body, and which concentrates in body fluids such as the cardiovascular system, to exert a remote regulatory effect on any organ or tissue in the body, or any artificial or exogenously administered chemical substances that may mimic or have similar effects to any naturally occurring chemical substance that circulates in body fluids and has a regulatory effect on any remote organ or tissue in the body.

An “insertion device” is a device which may assist with the insertion or placement of an endoprosthesis into position in or on the body of the subject. For example, an bronchoscope may be used as an insertion device for the placement of a bronchial stent; and endoscope or nasogastric tube may be used as an insertion device during placement of a gastrointestinal endoprosthesis, or a vascular catheter may be used as an insertion device for the placement of a vascular stent.

“Vascular” means pertaining or related to blood vessels or lymphatics.

“Stent” means a substantially hollow endoprosthesis that is inserted into a blood vessel, lymphatic, or body passage, or applied externally to a blood vessel, lymphatic or body passage, to keep its lumen open, or to prevent its closure or narrowing due to stricture, external compression, or other cause.

A “therapeutic substance” is any substance that has or is intended to have a salutary, favorable, disease-fighting, or health-promoting effect on any organism, organ system, organ, tissue, cell, or subcellular organelle in the body.

“Vasoactive agent” means any substance that directly or indirectly enhances or reduces the tone, tension, or degree of contraction of any blood vessel or lymphatic.

“Vibration” is a process of oscillation, or an alternating or reciprocating motion, generally about a position of equilibrium. Vibration is generally interpreted as cyclical (symmetrical or nonsymmetrical) fluctuations in the rate at which an object accelerates. In longitudinal vibration or longitudinal oscillation, the direction of motion of the oscillating body is the same as the direction of advance of the vibratory motion; in transverse vibration or transverse oscillation, the direction of motion of the oscillating body is perpendicular to the direction of advance. 13y oscillation is meant a fluctuation on one side of a mean position. Vibration may be partially described by the number of oscillations per period of time. In general, two oscillations about a mean position may be considered a cycle. One cycle per second is 1 Hertz (Hz).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including explanations of terms, will control. In addition, the materials, methods, and examples are illustrative only and are not intended to be limiting.

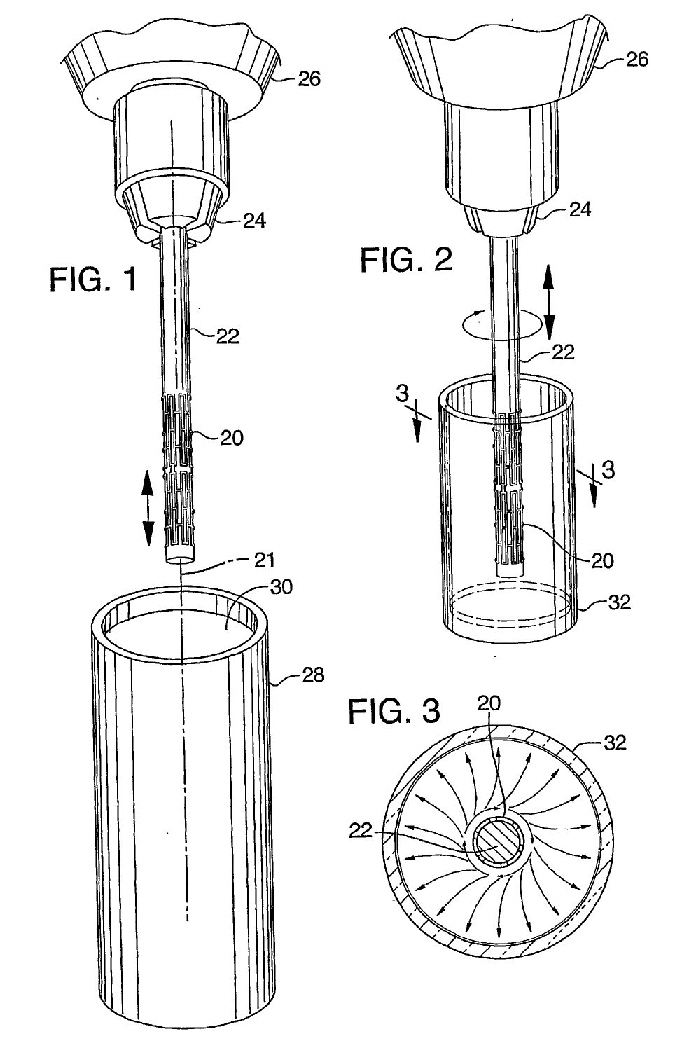

### EMBODIMENT OF FIGS. 1-3

A device for coating vascular stents is to shown in FIGS. 1-3. FIG. 1 shows a stent 20 mounted on an elongated cylindrical mandrel 22. Stent 20 as illustrated is a Palmaz-Schatz balloon-expandable intravascular stent available from Cordis Inc., Johnson & Johnson, Warren, N.J. The Palmaz-Schatz stent is 15 mm long and consists of two 7 mm slotted stainless steel tubes that are connected by a 1 mm central bridging strut. The unexpanded stent is 1.6 nm in diameter, and is substantially tubular and substantially symmetric about a longitudinal axis 21.

Mandrel 22 is rotatably connected by a chuck 24 to a motor 26. Chuck 24 is adjustable, to allow mandrels of different diameters to be engaged securely within the chuck. Stent 20 and mandrel 22 are shown poised above a vat 28 containing a bioactive liquid 30. Vat 28 has an inner diameter greater than stent 20 and mandrel 22, and is typically capable of holding about 0.5 ml to about 500 ml of bioactive liquid.

FIG. 2 illustrates stent 20 and mandrel 22 surrounded by a tubular shield 32. Shield 32 has an inner diameter greater than an outer diameter of stent 20 and mandrel 22, to allow the stent and mandrel to be inserted into shield 32. FIG. 3 is a cross-section of FIG. 2 at the level indicated by view line 3-3 in FIG. 2; it shows stent 20 and mandrel 22 surrounded by shield 32.

In operation, as shown in FIG. 1, stent 20 and mandrel 22 are introduced into vat 28 to immerse stent 20 in bioactive liquid 30 contained in vat 28. Stent 20 and mandrel 22 are then withdrawn from bioactive liquid 30 and inserted into shield 32 as illustrated in FIG. 2. Once stent 20 is surrounded by shield 32, motor 26 rotates chuck 24, mandrel 22 and stent 20 in a manner that generates enough centrifugal force to remove excess bioactive liquid 30 from stent 20. FIG. 3 illustrates this step, in which droplets of liquid are thrown off the surface of stent 20 as it rotates, and may collect on a closed bottom or inner walls of shield 32. Excess bioactive liquid 30 collected on the closed bottom or inner walls of shield 32 may be reused for stent coating or other purposes.

Although FIG. 2 illustrates stent 20 and mandrel 22 inserted into shield 32 from above, one skilled in the art would recognize that they can also be inserted from below, from the side, or indeed from any orientation. One skilled in the art would also recognize that the illustrated assembly of mandrel 22, chuck 24, and motor 26 represents one of many kinds of apparatuses capable of delivering centrifugal force. Numerous alternative methods and devices for delivering centrifugal force would be suitable in the practice of this invention. In addition, although clockwise rotation is illustrated, counterclockwise rotation would also be effective. In other embodiments the shield may not be used.

As one alternative to centrifugal force, vibrations may be applied to the endoprosthesis, for example by a mechanical vibrator. For example, mandrel 22 may be operably connected to a motor which delivers vibrations. The vibrations may be delivered at a broad range of frequencies which may be subsonic (from about 10 to about 40 Hz), in the sonic range (from about 40 Hz to about 20,000 Hz), or ultrasonic range (from about 20,000 Hz to about 200,000 Hz). For example, vibrations may be applied at about or more than any of the following frequencies: 10 Hz, 20 Hz, 50 Hz, 100 Hz, 200 Hz, 400 Hz, 1000 Hz, 2000 Hz, 5000 Hz, 10,000 Hz, 20,000 Hz, 40,000 Hz, 100,000 Hz, 200,000 Hz, or greater. The vibrations may be delivered to the catheter-stent assembly at, about, or more than any of the following time periods: 0.01 seconds, 0.1 seconds, 0.5 seconds, one second, two seconds, five seconds, 10 seconds, 20 seconds, 40 seconds, 1 minute, 2 minutes, 5 minutes, 10 minutes, 20 minutes, 40 minutes, 2 hours, 5 hours, 10 hours, 1 day, 2 days, 1 week, 2 weeks, or longer. Vibrations may be longitudinal oscillations or transverse oscillations, or any combination. The optimal characteristics of the vibrations—for example, they are frequency, amplitude, direction, and duration—is usually determined empirically, and depends in part on the size, shape and composition of the endoprosthesis, the viscosity of the bioactive liquid, surface tension of the bioactive liquid, and other factors. The characteristics of the vibrations may be varied in any manner during application of vibratory force.

By lengthening the walls of vat 28, stent 20 and mandrel 22 could be immersed in bioactive liquid 30 contained in vat 28, raised above the liquid, and rotated while stent 20 remained surrounded by the walls of vat 28. This would obviate the use of shield 32, because the shield's function would be served by vat 28 itself.

To generate sufficient centrifugal force to remove excess bioactive liquid, the stent may be rotated within a broad range of speeds, for example 100-100,000 RPM, 1,000-50,000 RPM, or 10,000-35,000 RPM. The optimal speed of rotation is usually determined empirically, and depends in part on the size and shape of the endoprosthesis, the viscosity of the bioactive liquid, surface tension, and other factors. If necessary or useful, the speed of rotation may be varied in any manner during rotation, depending on the size and shape of the endoprosthesis, the viscosity of the bioactive liquid, surface tension, and other factors.

The composition of bioactive liquid 30, and therefore its physical characteristics such as viscosity, will vary depending on a number of factors. These factors include the solubility of the desired therapeutic substance in various solvents, the desired thickness of the coating, the desired duration for delivery of the therapeutic substance, and other factors. The methods of this invention are not limited by viscosity, and would work with very low viscosity bioactive liquids (for example, a therapeutic substance dissolved in acetone), intermediate viscosity bioactive liquids (for example, a therapeutic substance dissolved in ethanol), or high viscosity bioactive liquids (for example, a polymer composition such as polycaprolactone).

The bioactive liquid may be a volatile bioactive liquid containing a biocompatible material or therapeutic substance. For example, the bioactive liquid may be a solution or suspension of a therapeutic substance dissolved in or suspended in ethanol, other alcohols, acetone, and the like. Such volatile liquids are particularly suitable for use with distributive forces such as centrifugal force and vibration, for example as described in Examples 2-5 herein.

It is observed that the use of volatile liquids improves uniformity of distribution and deposition of therapeutic substances such as paclitaxel and its derivatives. The improved uniformity is particularly apparent when the bioactive and/or therapeutic material being deposited is hydrophobic. Many therapeutic agents are substantially hydrophobic and soluble in volatile liquids such as ethanol. The methods disclosed herein are particularly suitable for such combinations of volatile liquids and substantially hydrophobic therapeutic agents.

Without wishing to commit themselves to any particular mechanism, the inventors currently believe that the application of distributive force may improve distribution and deposition by enhancing bonding of therapeutic substances (particularly hydrophobic therapeutic substances in volatile solvents) to the surface of endoprostheses.

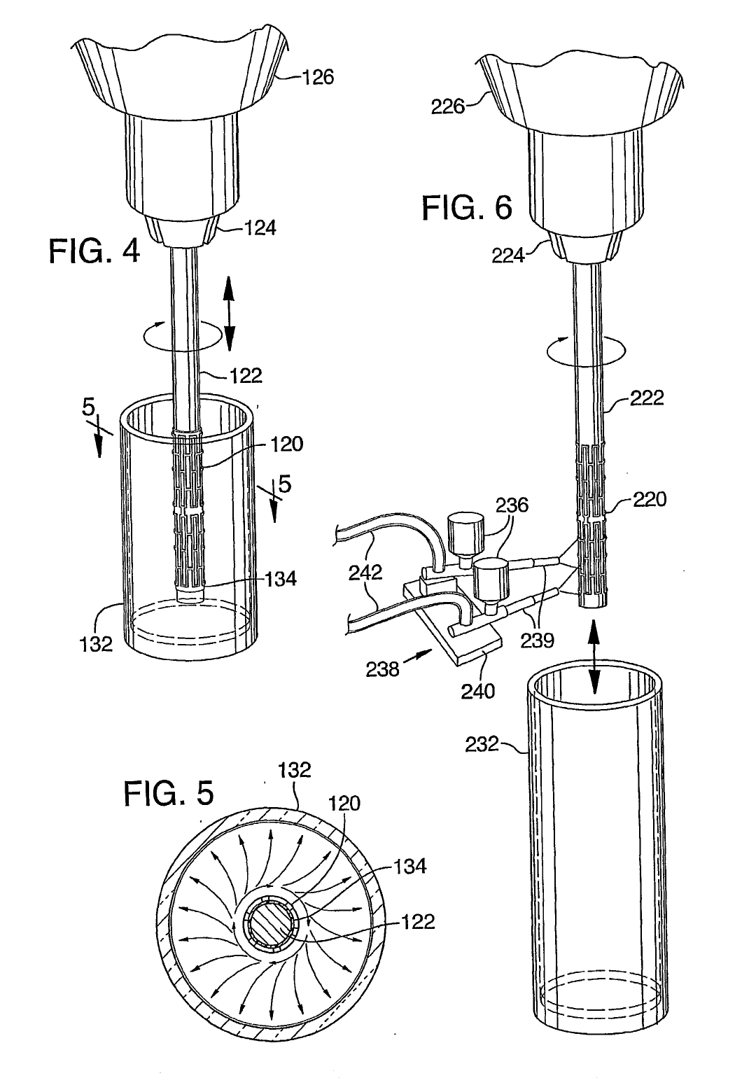

### EMBODIMENT OF FIGS. 4-5

Another embodiment is shown in FIGS. 4-5. In FIGS. 4-5, parts that correspond to those shown in FIGS. 1-3 are given corresponding numbers, plus 100. FIG. 4 shows a stent 120, a mandrel 122, a chuck 124, and a motor 126, and in these respects the embodiment is identical to that shown in FIGS. 1-3. In FIG. 4, however, stent 120 is not directly mounted on mandrel 124 as it was in FIGS. 1-3. Instead, a tubular sleeve 134-is mounted on mandrel 122, and stent 120 is mounted on sleeve 134. The sleeve may be made of any suitable material that functionally or otherwise engages the surrounding stent. Examples of such materials include silicone rubber, natural rubber, polyvinylchloride, polyurethanes, polyesters, polyethylene, polytetrafluoroethylene (PTFE), and the like.

Sleeve 134 may enhance the fit between stent 120 and mandrel 122. When positioned on mandrel 122, outer diameter of sleeve 134 is typically slightly smaller than the inner diameter of stent 120. For example, the Palmaz-Schatz stent illustrated in this embodiment has an unexpanded inner diameter of about 1.6 mm; an example of a sleeve that could be used with the stent would be a sleeve having an outer diameter of about 1.4 mm to 1.59 mm when mounted on mandrel 122. Stents may have considerably larger diameters, and in such instances, the sleeve could have a larger outer diameter. For example, the Palmaz Corinthian transhepatic biliary Stent (Cordis Inc., Johnson&Johnson, Warren, N.J.) may have an inner diameter of about 6 mm; an example of a sleeve for this stent would be a sleeve having an outer diameter of about 5.8 mm to about 5.99 mm when mounted on mandrel 122.

Sleeve 134 may also limit the amount of bioactive liquid on the surface of the stent in contact with the sleeve. Such limitation may be desirable in some circumstances. For example, it may be desirable to limit the amount of bioactive liquid and/or therapeutic material deposited on the lumenal surface of a stent (that is, the interior surface of the stent, the surface that faces the lumen of an organ or blood vessel when the stent is properly positioned in the body). Limiting lumenal deposition of bioactive liquid may enhance precision and/or accuracy of drug delivery, or more definitively target a therapeutic substance to the wall of an organ.

In operation, the embodiment of FIGS. 4-5 is similar to the embodiment of FIGS. 1-3. In FIGS. 4-5, sleeve 134 is mounted on mandrel 122, and stent 120 is mounted on sleeve 134. Stent 120 is immersed in a bioactive liquid and removed from the bioactive liquid (not shown). Shield 132 is placed around stent 120. A motor 126 rotates a chuck 124, mandrel 122, sleeve 134, and stent 120 in a manner that generates sufficient centrifugal force to remove excess bioactive liquid from stent 120. Excess bioactive liquid is collected on the inner walls or closed bottom of shield 132.

### EMBODIMENT OF FIG. 6

FIG. 6 illustrates applying a bioactive liquid to the stent by spraying the liquid onto the stent prior to or simultaneous with, the application of the distributing force. Spraying liquid on a stent is one of several alternatives for covering a stent with a bioactive liquid or polymer, and is exemplified by U.S. Pat. No. 5,980,972.

In FIG. 6, parts that correspond to those shown in FIGS. 1-3 are given corresponding numbers, plus 200. A stent 220 is mounted on a mandrel 222, rotatably connected by a chuck 224 to a motor 226. Two spray canisters 236 containing a bioactive liquid are mounted on a spray nozzle assembly 238, leaving a pair of spray nozzles 239 directed at mandrel 222, or stent 220 mounted on the mandrel. A structural support 240 supports spray nozzle assembly 238. Two pneumatic hoses 242 are connected on one end to spray canisters 236, and on the other end to a source of compressed air or compressed nitrogen (not shown).

In operation, compressed air drives bioactive liquid from spray nozzle assemblies 238 toward stent 220, as mandrel 222 is slowly rotated (e.g., 2-200 RPM), to coat stent 220 with the bioactive liquid. Once stent 220 is covered with bioactive liquid, stent 220 is surrounded by a shield 232. A motor 226 rotates chuck 224, mandrel 222, and stent 220 in a manner that generates sufficient centrifugal force to remove excess bioactive liquid from stent 220. Excess bioactive liquid is collected on the inner walls or covered bottom of shield 232.

One skilled in the art would recognize that there are many methods for applying liquid to an endoprosthesis, such as immersion, spraying, pouring, dripping, etc.

In the embodiments shown in FIGS. 1-6, the endoprosthesis is a substantially tubular stent that is mounted on a mandrel. However, the methods exemplified need not be confined to tubular endoprostheses. For example, a solid endoprosthesis could be securely attached to a mandrel, coated with a liquid, and the mandrel spun to generate centrifugal force and thereby remove excess liquid.

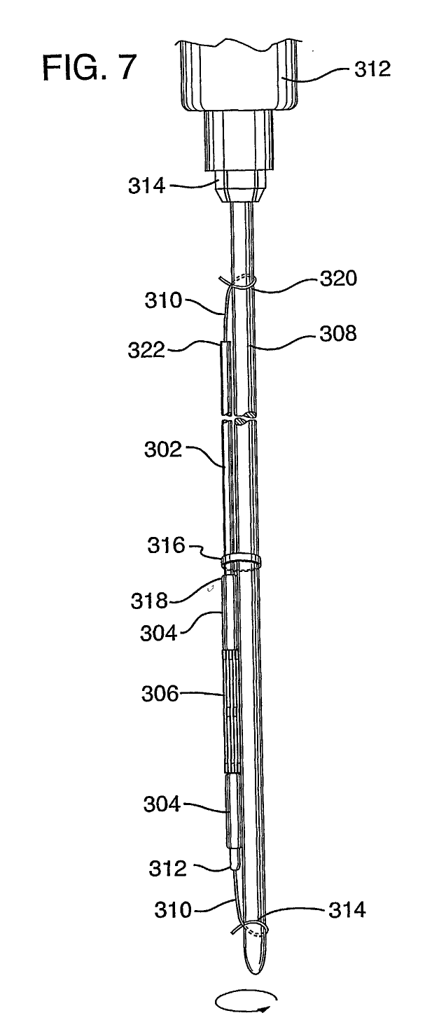

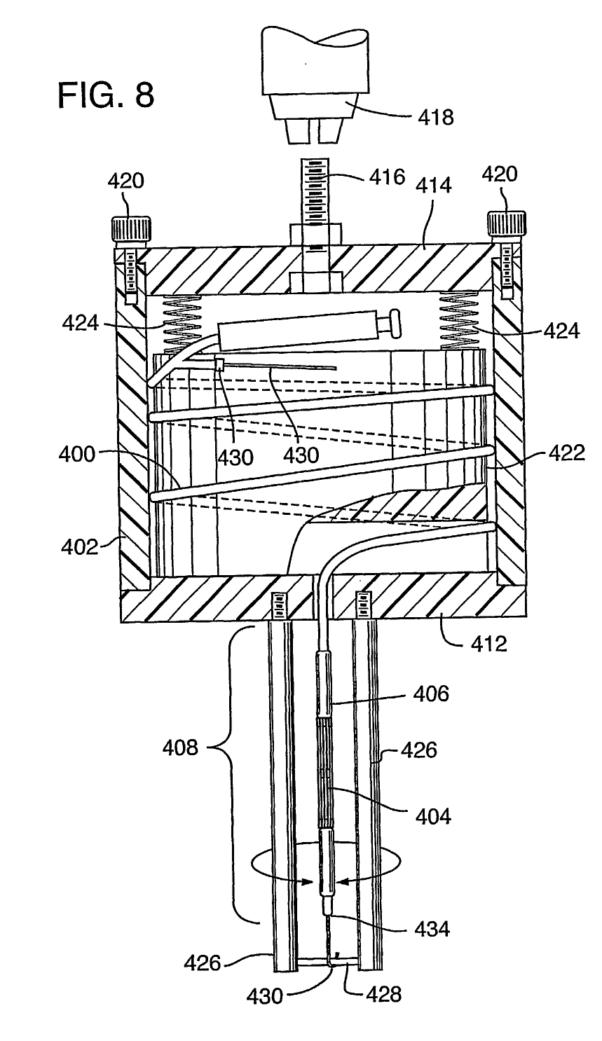

### EMBODIMENTS OF FIGS. 7-8

FIGS. 7-8 illustrate that a bioprosthesis may be covered with a bioactive liquid after it is mounted on an inserting device. This provides the advantage of reducing manipulation and handling of the bioprosthesis after it has been coated. Such handling may lead to loss of the therapeutic substance from the bioprosthesis, producing undesirable therapeutic variability or therapeutic failure. FIG. 7 illustrates a catheter-stent assembly, comprising a hollow, substantially tubular vascular catheter 302 having a central lumen, a balloon tip 304, and a stent 306 which is mounted on the balloon tip. The catheter-stent assembly is affixed to a rotatable spool 308 via a wire 310 that is threaded through the length of the catheter's central lumen and emerges from a hole 312 in the catheter's distal end. Wire 310 is tied or in some manner attached to spool 308 at a point of attachment 314. The catheter stent assembly may also be attached to spool 308 at an additional point of attachment 316. By way of example, additional point of attachment 316 is illustrated as being proximal to a proximal end 318 of balloon 304. A third point of attachment 320 maybe at proximal end of wire 310 as it emerges from the catheter's proximal end 322. Multiple additional points of attachment are possible as well, to achieve one objective of reasonably securing the catheter-stent assembly to spool 308 as rotational force is applied.

Stent may be epicentric to the spool's axis of rotation, as illustrated in FIG. 7, or it may be centered about the spool's axis of rotation. One approach to centering the catheter-stent assembly about the axis of rotation is to use a wire extending through the catheter's central lumen as the spool. Rotational force is applied externally to the catheter, for example by rotating a platform on which the entire assembly rests or applying external rotational force at various points along the length of the catheter (for example, with spinning circular wheels whose axis of rotation is about perpendicular to the catheter's long axis, and which contact the catheter, causing it to spin relative to the spool).

Force may be applied to spool 308 by a number of means. For example, spool 308 may be a mandrel rotatably connected via a chuck 324 to a power motor, in a manner similar to that illustrated and described in FIGS. 1-3 and accompanying text. Suitable power motors are commercially manufactured by several manufacturers, for example Sears, Roebuck & Co, Chicago Ill. or Dremel Inc., Racine Wis.

Alternatively, the mandrel may operably connected to a motor which delivers vibrations to the spool and attached catheter stent assembly. The vibrations may be delivered at a broad range of frequencies, for example, at about or more than any of the following frequencies: 5 Hz, 20 Hz, 50 Hz, 100 Hz, 200 Hz, 400 Hz, 1000 Hz, 2000 Hz, 5000 Hz, 10,000 Hz, 20,000 Hz, or greater. The vibrations may be delivered to the catheter-stent assembly at, about, or more than any of the following time periods: 0.1 seconds, 0.5 seconds, one second, two seconds, five seconds, 10 seconds, 20 seconds, 40 seconds, 1 minute, 2 minutes, 5 minutes, 10 minutes, 20 minutes, 40 minutes, 2 hours, 5 hours, 10 hours, 1 day, 2 days, 1 week, 2 weeks, or longer. Vibrations may be longitudinal vibrations or transverse vibrations, or any combination.

FIG. 8 illustrates a catheter-stent assembly in which a portion of a catheter 400 is contained within a catheter containment device 402. Catheter containment device 402 serves to restrain catheter 402 and stent 404 as centrifugal, vibratory, or other distributive force is applied to the catheter-stent assembly.

In a particular illustrated embodiment, catheter containment device 402 is approximately cylindrical and constructed of a translucent polymer such as Lexan. However, catheter containment device may be constructed of virtually any durable material, such as polypropylene, polyethylene, other plastics, aluminum or other metal, wood, or the like. Although illustrated as cylindrical, catheter containment device 402 may be cylindrical, or may be virtually any noncylindrical shape.

Catheter stent assembly includes a substantially tubular catheter 400 having a balloon tip 406 and a distal end 408. The catheter stent assembly may also include a mounted stent 404, which is mounted upon balloon tip 406. Catheter containment device 402 has an opening 410 on a bottom surface 412. Opening 410 is sufficiently large to allow a catheter and stent to pass through it.

An upper surface 414 of catheter containment device 402 is attached to a mandrel 416. Mandrel 416 may be rotatably or vibratably connected to a power motor, for example through a chuck 418. Upper surface 414 maybe detachable, for example by removing one or more fasteners 420 and lifting upper surface 414 away from catheter containment device 402. Alternatively, upper surface 414 may have an opening through which a catheter may be inserted and positioned within the containment device. The opening may be sealable, for example with a stopper made of rubber, polypropylene, polyethylene, other plastics, or other suitable material.

Catheter containment device 402 may also contain material which helps to restrain catheter movement and/or protect the catheter from damage during application of distributive force. In FIG. 8, a cylindrical spool 422 is illustrated. Catheter 400 may be partially wound around spool 422. One skilled in the art would see that many materials are suitable for use as a spool, for example Styrofoam, foam rubber, lightweight plastic, wood or metal covered with a soft material such as air bubble packing material, and the like. Winding of the catheter around the spool is not essential. Nor is a cylindrical or spool shape essential. For example, the catheter containment device may be partially or substantially filled with Styrofoam peanuts, shredded newspaper, or packing material having multiple air bubbles. Alternatively, such material may simply be omitted.

The position of spool 422 may be optionally stabilized by one or more motion dampers 424, for example by one or more springs or the like. Upon placing top surface 414 into position, motion dampers 424 engage spool 422 to prevent excessive movement of spool during application of distributive force to the catheter containment device. Numerous other motion damper arrangements are possible, or the motion dampers may simply be omitted.

One or more stabilizers 426 are attached to lower surface 412 of catheter containment device 402. The stabilizer 426 may be a single piece, for example a cylindrical piece of thin metal or plastic, or may be a plurality of stabilizers, for example two or more parallel bars or rods extending from lower surface 412. The stabilizer 426 has attached thereto a pin 428, which may connect two approximately opposing sides of the stabilizer.

Catheter 400 may contain a wire 430 which emerges from catheter 400 at distal end 408 and a proximal port 432. Wire 430 is slidable within catheter 400, and may be completely removed by pulling it proximally out proximal port 430, or pulling it distally through a hole 434 at the distal end of catheter 400.

In operation, catheter 400 is placed into catheter containment device 402, and its distal end 408 guided through opening 410 on bottom surface 412 of catheter containment device 402. If desired, catheter may be partially or completely wrapped around spool 422, or other material may be added to the catheter containment device to absorb motion within the catheter containment device. Wire 430 is placed in catheter 400 and its distal end attached to pin 428.

Rotation of a cylindrical catheter containment device with catheter substantially wrapped around a cylindrical spool has the favorable effect of enhancing stability during rotation. In addition, approximately centering the stent about the axis of rotation may improve the uniformity of distribution of the bioactive liquid on the stent. Such an arrangement is illustrated in the embodiment of FIG. 8.

Distal end 408 of catheter 400 and (if present) stent 404 are coated with a bioactive liquid by immersion, spraying, or other means as described herein. During and/or after application of bioactive liquid, centrifugal, vibratory, or other distributive force is applied to catheter containment device 402. The nature of the distributive force, amount of distributive force, its duration, are determined empirically in manner similar to that described in the embodiments of FIGS. 1-6.

After application of distributive force to the catheter containment device, catheter stent assembly is removed from catheter containment device 402. This may be accomplished by removing top surface 414, removing wire 430 from catheter 400 (for example by pulling proximally through proximal port 432), removing catheter 400 from catheter containment device by advancing its distal end 408 through opening 410 (so that distal end 408 is at least briefly contained within, or passes through, the catheter containment device). The catheter or catheter and mounted stent may then be removed for use, or for storage and later use.

If desired, catheter stent assembly may be sterilized prior to use in a subject, for example by exposure to ethylene oxide. Such sterilization techniques are well known to those skilled in the art.

The embodiment of FIG. 7 and/or FIG. 8 may be combined with one or more of the features illustrated in FIGS. 1-6. For example, the catheter-stent assembly of FIG. 7 or FIG. 8 may be coated with a bioactive liquid by immersing into a vat, removing the catheter stent assembly from the vat, and applying centrifugal force to the catheter stent assembly, as illustrated in FIGS. 1-4. The catheter-stent assembly may be surrounded by a shield that collects excess bioactive liquid as centrifugal force is applied to the catheter-stent assembly. The bioactive liquid may be sprayed on the catheter-stent assembly, as illustrated in FIG. 6.

### EXAMPLE 1

### Manual Coating

In this example, Palmaz-Schatz stents were coated with paclitaxel using a manual method. The amount of paclitaxel deposited was then determined.

Stents were coated by the manual method as follows: each stent was held by a pair of forceps and dipped into one of five different reservoirs containing 95% ethanol and varying concentrations of paclitaxel (13 mg/ml; 27 mg/ml; 37 mg/ml; 42 mg/ml; 66 mg/ml). Each stent was then removed from the reservoir, shaken briskly, and allowed to dry. The amount of paclitaxel deposited on each stent was determined by immersing the stent in 1 ml of 95% ethanol, removing the stent, and then analyzing the amount of paclitaxel in the 95% ethanol solution using a Gilson modular High Pressure Liquid Chromatograph (HPLC). Ultraviolet detection at 227 nanometers (Gilson 116 detector) was utilized to quantitate paclitaxel after elution from a 25 cm×4.6 mm pentafluorophenyl column (ES Industries). The solvent system consisted of 45% acetonitrile and 55% water delivered by a pair of Gilson 305/306 pumps with a flow rate of 1.5 ml/minute.

TABLE 1

| Paclitaxel | Amount of drug deposited | Number of stents |
|---|---|---|
| concentration | (mean ± standard deviation) | in group |
| 13 mg/ml | 19.5 ± 7.7 μg | 16 |
| 27 mg/ml | 59.8 ± 17.6 μg | 11 |
| 37 mg/ml | 80.4 ± 27.7 μg | 12 |
| 42 mg/ml | 141.3 ± 43.0 μg | 24 |
| 66 mg/ml | 198.3 ± 72.0 μg | 16 |

These results emphasize the difficulty of controlling drug deposition on stents when the manual coating method is used. First, there is a relatively weak and nonlinear correlation of drug deposition with paclitaxel concentration. Second, the high standard deviations shows that drug deposition from stent-to-stent is highly variable.

### EXAMPLE 2

### Coating Using Centrifugal Force

In this example, Palmaz-Schatz stents were coated with paclitaxel using a method of spin-coating that applied centrifugal force to the stents to distribute the drug on their surfaces. The amount of paclitaxel deposited was then determined.

Stents were mounted on the mandrel of a Dremel high speed professional rotary tool (Dremel Inc., Racine Wis.), then immersed in one of four different reservoirs containing 95% ethanol and varying concentrations of paclitaxel (18 mg/ml; 28 mg/ml; 37 mg/ml; 54 mg/ml). The stent-mandrel assembly was then removed from the reservoir and inserted into a shield. The Dremel tool was then used to spin the stent-mandrel assembly at 20,000-22,000 RPM for 1-2 seconds. The stent was allowed to dry, and was then carefully removed from the mandrel. The amount of paclitaxel deposited on each stent was determined using HPLC as described in Example 1.

The data from this analysis are presented in Table 2, below.

TABLE 2

| | Amount of drug deposited | Number of stents |
|---|---|---|
| Paclitaxel concentration | (mean ± standard deviation) | in group |
| 18 mg/ml | 6.1 ± 0.5 μg | 4 |
| 28 mg/ml | 13.5 ± 1.4 μg | 3 |
| 37 mg/ml | 18.6 ± 2.9 μg | 6 |
| 54 mg/ml | 23.1 ± 2.8 μg | 5 |

These results show that using centrifugal force in stent-coating protocols leads to excellent control of drug deposition. First, there is a tight linear correlation between paclitaxel concentration and drug deposition. Second, the standard deviations in each group are substantially smaller than the drug-coated stents of Example 1, establishing that use of centrifugal force markedly enhances reproducibility of drug deposition. Additional experiments revealed that a Craftsman motor tool (Sears, Roebuck & Co, Chicago Ill.) run at 12,000 RPM for two seconds also yielded satisfactory results.

### EXAMPLE 3

### Uniformity of Drug Deposition

To evaluate uniformity of drug deposition, photomicrographs were obtained of spin-coated stents and stents coated with the manual method of Example 1.

Manually coated stents exhibit significant flaws that are inherent in the manual coating method. First, the coating is substantially uneven; second, cracks in the coating are clearly visible; third, there are areas where the coating has almost completely flaked off the stent. In contrast, the spin-coated stent shows uniform drug deposition without evidence of cracking or flaking.

Fluorescence photomicrographs of manually coated Palmaz-Schatz stents revealed substantially uneven coating. There were large areas that are devoid of any drug coating. Cracks in the coating are clearly visible. In contrast, spin coated stents demonstrated substantially uniform fluorescence, substantially complete coverage, and the absence of cracks.

This evidence establishes that the use of centrifugal force markedly enhances uniformity of drug deposition.

### EXAMPLE 4

### Effect of Crimping

Prior to being deployed inside a blood vessel, some vascular stents must be crimped onto a balloon-tipped catheter. Once the stent is positioned on the catheter, the catheter-stent assembly is advanced in the blood vessel to the site of obstruction. The balloon is then expanded, which deploys the stent in its proper position.

Since the manual coating method leads to uneven, fragile coating (see Examples 1-3), it seemed likely that the process of crimping would exacerbate the problem of flaking and drug loss. To demontstrate this problem, the amount of paclitaxel on stents before and after crimping was determined. Stents were coated by the manual coating method as in Example 1: each stent was held by a pair of forceps and dipped into one of three different reservoirs containing 95% ethanol and varying concentrations of paclitaxel (37 mg/ml; 42 mg/ml; 66 mg/ml). Each stent was then removed from the reservoir, shaken briskly, and allowed to dry. The amount of paclitaxel deposited on each stent was determined using HPLC as described in Example 1.

The data from this analysis are presented in Table 3, below.

TABLE 3

| Paclitaxel | Amount of drug—crimped |
|---|---|
| Concentration | (mean ± standard deviation) |
| 37 mg/ml | 57.9 ± 33.0 μg |
| 42 mg/ml | 127.1 ± 34.6 μg |
| 66 mg/ml | 106.3 ± 53.8 μg |

These data establish that crimping of manually coated stents leads to very high standard deviations in amount of paclitaxel remaining on the stent. This establishes that the drug loss from manually coated stents is highly variable and unpredictable. Moreover, the loss was substantial; manually coated stents lost about 40% of the total amount of paclitaxel coating upon crimping. In contrast, Table 4 shows that spin-coated stents lost only a small and reproducible amount of drug upon crimping.

TABLE 4

| Paclitaxel | | |
|---|---|---|
| Con- | Amount of drug—uncrimped | Amount of drug—crimped |
| centration | (mean ± standard deviation) | (mean ± standard deviation) |
| 18 mg/ml | 6.1 ± 1.1 μg | 6.4 ± 2.1 μg |
| (n = 4 each) | | |
| 28 mg/ml | 13.5 ± 1.4 μg | 12.6 ± 1.2 μg |
| (n = 3 each) | | |

Thus, spin-coated stents retained more of their coating upon crimping, and had more uniform and predictable losses upon crimping, than did manually coated stents.

### EXAMPLE 5

### Effect of Stent Expansion

After a stent is crimped onto a catheter as in example 4, it is ready for deployment in the body. The stent is properly positioned with a catheter, and is then expanded, for example by inflation of a balloon tip around which the stent is mounted. The balloon inflation expands the stent and forces it into proper position in the wall of the hollow organ. This has an additional physical impact on the stent, and has the potential to further degrade its coating.

To demonstrate the impact of stent expansion on coating, Palmaz-Schatz stents were coated by immersion in a 44 mg/ml ethanolic solution of paclitaxel, then removed and subjected to centrifugal force (12,000 RPM for two seconds using a Craftsman motor tool). They were then crimp-mounted on a balloon-tipped catheter, expanded in a beaker of water, removed from the water and analyzed for paclitaxel content by HPLC as described in Example 1. Nine unexpanded stents had a mean paclitaxel amount of 32.8±5.9 μg/stent, whereas the expanded stent had 26.9 μg of paclitaxel. Thus, spin-coated stents respond well to expansion, with only a small amount of drug loss associated with expansion. In addition, 3 Palmaz-Schatz stents spin coated with a paclitaxel-Texas Red conjugate were crimp-mounted on a balloon-tipped catheter, expanded in a beaker of water, removed from the water, and examined under epiflourescence microscopy. The fluorescence was uniform across each stent's surface. Moreover, the three stents had quantitatively similar amounts of fluorescence. Thus, drug distribution on spin-coated stents remains uniform after expansion.

In view of the many possible embodiments to which the principles of the invention may be applied, it should be recognized that the illustrated embodiments are examples of the invention, and should not be taken as a limitation on the scope of the invention. Rather, the scope of the invention is defined by the following claims. We therefore claim as our invention all that comes within the scope and spirit of these claims.

1. (Reiterated) A method of coating an endoprosthesis, comprising:
applying a liquid to a surface of the endoprothesis; and
applying to the endoprothesis a distributive force that distributes the liquid substantially evenly over the surface of the endoprosthesis.

2. (Reiterated) The method of claim 1, wherein the surface of the endoprothesis is at least an outer surface of the endoprosthesis, and the distributive force is centrifugal force or vibration.

3. (Reiterated) The method of claim 2, wherein the distributive force is centrifugal force.

4. (Reiterated) The method of claim 2, wherein the distributive force is vibration.

5. (Reiterated) The method of claim 2, wherein the centrifugal force is directed only away from the surface of the endoprosthesis.

6. (Reiterated) The method of claim 1, wherein the endoprosthesis by rotating along a longitudinal axis, and the centrifugal force is applied to the endoprosthesis by rotating the elongated endoprosthesis substantially around the longitudinal axis.

7. (Reiterated) The method of claim 1, wherein the endoprosthesis is elongated along a longitudinal axis, and the vibration is applied to the endoprosthesis by vibrating the elongated endoprosthesis with longitudinal oscillation or transverse oscillation.

8. (Reiterated) The method of claim 1, further comprising providing a shield that surrounds the endoprosthesis while centrifugal force or vibration is applied to the endoprosthesis.

9. (Reiterated) The method of claim 8, wherein the shield collects liquid that is displaced from the endoprosthesis when the centrifugal force or vibration is applied.

10. (Reiterated) The method of claim 9, further comprising subsequently applying the liquid that is collected from the shield to the same or another endoprosthesis.

11. (Reiterated) The method of claim 8, wherein the shield is a tubular memeber.

12. (Reiterated) The method of claim 1, wherein the liquid comprises a therapeutic substance.

13. (Reiterated) The method of claim 12, wherein the therapeutic substance is selected from the group consisting of antithrombotic agents, antiplatelet agents, anti-inflammatory agents, antibotics, anti-angiogenic agents, angiogenesis-promoting agents, antioxidants, antiproliferative agents, anti-atherogenic agents, vasoactive agents, photoactivated agents, photosensitive agents, radiation sensitizing agents, acoustic energy sensitizing agents, radioactive agents, imaging agents, and hormones.

14. (Reiterated) The method of claim 13, wherein the therapeutic substance comprises a taxane.

15. (Reiterated) The method of claim 14, wherein the taxane comprises paclitaxel, or an analog thereof.

16. (Reiterated) The method of claim 1, wherein the endoprosthesis is a stent.

17. (Reiterated) The method of claim 16, wherein the stent is mounted on an insertion device prior to applying the liquid.

18. (Reiterated) The method of claim 16, wherein the endoprosthesis is a vascular stent.

19. (Reiterated) The method of claim 18, wherein the insertion device is a vascular catheter.

20. (Reiterated) The method of claim 18, wherein the vascular catheter has an inflatable distal end upon which the stent is mounted.

21. (Reiterated) The method of claim 19, wherein the vascular catheter is attached to a mandrel which is rotated or vibrated to distribute the liquid on the surface of the endoprosthesis.

22. (Reiterated) The method of claim 20, wherein at least part of the vascular catheter is placed within a catheter containment device, and the catheter containment device is rotated or vibrated.

23. (Amended) The method of claim 6, wherein the endoprosthesis is substantially symmetric with respect to the longitudinal axis.

24. (Amended) The method of claim 6, wherein the endoprosthesis is substantially tubular.

25. (Reiterated) The method of claim 1, wherein the distributive force is centrifugal force or vibration and the distributive force is applied by inserting a mandrel into the endoprosthesis, and rotating or vibrating the mandrel to rotate or vibrate the endoprosthesis.

26. (Reiterated) The method of claim 25, further comprising placing a sleeve between the mandrel and endoprosthesis, wherein the sleeve is of sufficient thickness to engage the endoprosthesis and rotate or vibrate the endoprosthesis with the mandrel.

27. (Reiterated) The method of claim 1, wherein the mandrel is a shaft to which the distributive force is applied by a motor.

28. (Reiterated) The method of claim 1, wherein applying the liquid to the surface of the endoprosthesis comprises immersing at least a portion of the endoprosthesis in the liquid.

29. (Reiterated) The method of claim 1, wherein applying the liquid to the surface of the endoprosthesis comprises spraying at least a portion of the endoprosthesis with the liquid.

30. (Reiterated) The method of claim 1, wherein a substantially same centrifugal force or a substantially same vibration is applied to multiple endoprostheses to improve uniformity of an amount of the liquid on the multiple endoprostheses.

31. (Reiterated) The method of claim 1, wherein the distributive force is centrifugal force, and the centrifugal force is applied by rotating the endoprosthesis at 100-100,000 RPM.

32. (Reiterated) The method of claim 31, wherein the endoprosthesis is rotated at 1,000-50,000 RPM.

33. (Reiterated) The method of claim 32, wherein the endoprosthesis is rotated at 10,000-35,000 RPM.

34. (Reiterated) The method of claim 1, wherein the distributive force is vibration, and the vibration is applied by vibrating the endoprosthesis at a frequency between about 10 Hz and about 200,000 Hz.

35. (Reiterated) The method of claim 34, wherein the endoprosthesis is vibrated at a frequency between about 20 Hz and about 100,000 Hz.

36. (Amended) The method of claim 35, wherein the endoprosthesis is vibrated at a frequency between about 40 Hz and about 100,000 Hz.

37. (Amended) The method of claim 35, wherein the endoprosthesis is vibrated at a frequency between about 50 Hz and about 40,000 Hz.

38. (Reiterated) The method of claim 34, wherein the endoprosthesis is vibrated at a frequency in the subsonic range.

39. (Amended) The method of claim 38, wherein the endoprosthesis is vibrated at at frequency in the sonic range or ultrasonic range. Please cancel claim 40.

40. (Amended) The method of claim 1, wherein the distributive force is centrifugal force or vibration, and a speed of rotation or frequency of vibration is substantially the same for different prostheses for which a substantially uniform amount of the liquid on the surface is desired.

41. (Reiterated) The method of claim 1, wherein a viscosity of the liquid is maintained substanially constant for different prostheses for which a substantially uniform amount of the liquid on the surface is desired.

42. (Amended) The method of claim 1, further comprising:
before, during or after applying the liquid, connecting the endoprosthesis to a driving member capable of applying the distributive force to the endoprosthesis;
applying distributive force to the endoprosthesis to distribute the liquid over the surface of the endoprosthesis.

43. (Reiterated) The method of claim 42, where the endoprosthesis is substantially symmetric, and applying distributive force to the endoprosthesis comprises rotating or vibrating the endoprosthesis around an axis of symmetry.

44. (Reiterated) The method of claim 42, wherein the driving member is a rotatable or vibratable driving member that is secured to the endoprosthesis.

45. (reiterated) The method of claim 44, wherein the rotatable or vibratable driving member is a mandrel that is inserted into the endoprosthesis.

46. (Reiterated) The method of claim 45, wherein the endoprosthesis is rotated to apply centrifugal force substantially transverse to the surface of the endoprosthesis.

47. (Reiterated) The method of claim 45, wherein the distributive force is vibration, and the vibration comprises longitudinal oscillations or transverse oscillations.

48. (Reiterated) The method of claim 44, wherein the endoprosthesis is a stent for placement in a biological lumen.

49. (Reiterated) The method of claim 48, wherein the stent is a vascular stent for placement in a vascular lumen, and the liquid is a therapeutic substance that helps maintain patency of the vascular lumen.

50. (Reiterated) The method of claim 42, wherein the method comprises providing a substantially uniform coating of the liquid on the surface of multiple similar endoprostheses.

51. (Reiterated) The method of claim 50, wherein the multiple endoprostheses are rotated at a similar speed of rotation, or the multiple endoprostheses are vibrated at similar frequencies.

52. (Amended) The method of claim 1 wherein the liquid is a therapeutic substance, the method further comprising:
immersing at least a portion of each of the stents in the therapeutic substance, wherein the therapeutic substance comprises a liquid of substantially unifor viscosity;
inserting a mandrel into each of the hollow stents, and securing each stent to the mandrel;and
rotating the stents at a substantially uniform speed of rotation that provides a substantially uniform coating of the therapeutic substance on the surface of the stents, or vibrating the stents at a frequency that provides a substantially uniform coating of the therapeutic substance on the surface of the stents. Please cancel claims 53-55

56. (Reiterated) A device for coating a surface of an endoprosthesis with a bioactive liquid, comprising:
a driver dimensioned and configured to hold an endoprosthesis while applying distributive force to the endoprosthesis;
a shield surrounding the driver collecting the bioactive liquid that leaves the surface of the endoprosthesis when applying distributive force to the endoprosthesis.

57. (Retierated) The device of claim 56, further comprising a source of the bioactive liquid.

58. (Reiterated) The device of claim 56, wherein the endoprosthesis is hollow and elongated along an axis of elongation, and the driver is a mandrel that is dimensioned for insertion into the hollow endoprosthesis.

59. (Reiterated) The device of claim 58, further comprising the endoprosthesis mounted on the driver.

60. (Reiterated) The device of claim 56, further comprising a spacer between the driver and endoprosthesis for maintaining engagement between the driver and endoprosthsis when rotating or vibrating the endoprosthesis.

61. (Reiterated) A coated endoprosthesis, wherein the endoprosthesis has been coated with a bioactive liquid by the method of claim 1.

62. (Reiterated) The coated endoprosthesis of claim 61, wherein the bioactive liquid is a therapeutic substance.

63. (Reiterated) The coated endoprosthesis of claim 61, wherein the coated endoprosthesis is a stent.

64. (Reiterated) The stent of claim 63, wherein the stent is a vascular stent.

65. (Reiterated) The stent of claim 63, wherein the stent is substantially hollow and elongated about an axis of elongation. Please cancel claim 66.
